(19) United States
(12) Patent Application Publication
(10) Pub. No.: US 2025/0072345 A1
(43) Pub. Date: Mar. 6, 2025

(54) METHODS FOR IMPROVING AGRONOMIC TRAITS OF A PLANT

(71) Applicant: The Board of Trustees of the University of Illinois, Urbana, IL (US)

(72) Inventor: Ken N. PAIGE, Monticello, IL (US)

(21) Appl. No.: 18/404,084

(22) Filed: Jan. 4, 2024

Related U.S. Application Data
(60) Provisional application No. 63/536,052, filed on Aug. 31, 2023.
(60) Provisional application No. 63/580,200, filed on Sep. 1, 2023.

(51) Int. Cl. A01H 1/00; A01H 1/06

(52) U.S. Cl. CPC A01H 1/1245; A01H 1/06

(57) ABSTRACT

Methods for improving one or more agronomic traits of a plant by selecting a plant variety that exhibits overcompensation is provided as is a method for producing one or more plant products from a modified plant that exhibits improvements in one or more agronomic traits. The methods may include reducing or inhibiting the biosynthetic production of one or more plant chemical defense compounds prior to removing the plant apical meristem at a time that is known to cause overcompensation in selected plants.

This application claims benefit from U.S. Provisional Patent Application Ser. No. 63/536,052, filed Aug. 31, 2023 and 63/580,200, filed Sep. 1, 2023, the contents of which are incorporated herein by reference in their entireties.

### INTRODUCTION

This invention was made with government support under grant no. DEB 1146085, awarded by the National Science Foundation. The government has certain rights in this invention.

### STATEMENT REGARDING ELECTRONIC FILING OF A SEQUENCE LISTING

A Sequence Listing in XML text format, submitted under 37 C.F.R. § 1.821-1.834, entitled “IL0043US,” 3,623 bytes in size, generated Dec. 26, 2023, is provided in lieu of a paper copy. This Sequence Listing is hereby incorporated by reference into the specification.

### BACKGROUND

Plants possess multiple types of defenses against herbivores, including constitutive and inducible chemical compounds, structural traits (collectively, resistance) and regrowth/fitness strategies (tolerance). These defense strategies typically co-occur within the same plant. Multiple defense mechanisms have been hypothesized to be costly for a plant, as investment in one anti-herbivore defense is assumed to reduce resources available for other defenses and growth and reproduction. Therefore, constraints on multiple defensive strategies due to resource allocation costs are predicted to manifest as a negative association between the defenses, however this is not always the case. Such a trade-off has been predicted to occur between plant tolerance (fitness compensation) and resistance chemistry. If a trade-off were to exist it would have important evolutionary ramifications because it would result in selection leading either to maximal resistance or maximal tolerance, but not both. However, a meta-analysis on plant resistance and tolerance showed most natural populations appear to be composed of a mixture of both strategies, possibly due to selection for the maintenance of both traits caused by the joint feeding of both generalist and specialist herbivores.

What has been missing from studies of potential trade-offs between tolerance and resistance is an understanding of the molecular genetic pathway involved in tolerance and its relationship to well-characterized molecular pathways involved in chemical defense, such as the shikimate pathway. Studies on the molecular underpinnings of tolerance (with an emphasis on the phenomenon of overcompensation) have shown that plant tolerance and resistance are positively correlated due to a sharing of the same molecular genetic pathways, explaining in part the inability to uncover a physiological trade-off between these two strategies (Siddappaji et al. (2013) Genetics 195:589-598; Mesa et al. (2017) Ecology 98: 2528-2537), yet acknowledging that trade-offs could still occur at lower levels of the trait hierarchy and manifest positively at higher levels. Specifically, glucose-6-phosphate-1-dehydrogenase (G6PD1, At5g35790.1) has been shown to play a major role in controlling the compensatory response in Arabidopsis thaliana following the removal of the plant's primary inflorescence (Siddappaji et al. (2013) Genetics 195:589-598). G6PD1 is the central regulatory enzyme in the oxidative pentose phosphate (OPP) pathway that plays a key role in plant metabolism generating NADPH and a variety of metabolic intermediates for biosynthetic processes including resistance to oxidative damage, the production of plant secondary defensive compounds, such as glucosinolates (of interest herein) and the production of ribo- and deoxyribonucleotides.

It has also shown that following the removal of apical dominance, phenotypically plastic increases in ploidy level via endoreduplication leads to rapid regrowth and an increase in fitness, explaining, in part, the phenomenon of overcompensation in plants (Scholes & Paige (2014) Molecular Ecology 23:4862-4870). Endoreduplication is the replication of the genome without mitosis, which leads to endopolyploidy, an increase in cellular chromosome number. Removal of the apical meristem by herbivores eliminates production of the plant hormone auxin, leading to a rapid break in dormancy of axillary buds and subsequent stem elongation. High levels of auxin are also known to repress the endocycle, and by contrast, lower levels of auxin trigger an exit from mitotic cycles and an entry into endocycles. Insect leaf-feeding also can trigger endoreduplication by the upregulation of jasmonic acid, which also lowers auxin production and can lead to overcompensation in some ecotypes of Arabidopsis (Mesa et al. (2019) Oecologia 190:847-856). Thus, there is a direct link between endoreduplication and plant damage.

Increasing chromosome number through endoreduplication and therefore gene copy number provides a means of increasing expression of vital genes such as G6PD1 or genetic pathways that promote rapid regrowth rates following herbivory. G6PD1 feeds compounds into the OPP pathway for nucleotide biosynthesis, by the provision of ribose-5-phosphate, necessary for the significant increase in chromosome number via endoreduplication. The increase in DNA content then feeds back positively on pathways involved in metabolism (e.g., G6PD1) and defense (e.g., glucosinolate production) through increased gene expression (more copies due to increases in endoreduplication following damage. An increase in total cellular DNA content through endoreduplication also leads to extensive cell growth via cell expansion. Rapid growth and development following the removal of apical dominance may be enhanced by maximizing nutrient transport (with fewer plasmodesmata), protein synthesis (with more copies of DNA), photosynthetic rates (with an increase in the number of chloroplasts) and light and water absorption (with larger cell size and storage capacity). Importantly, the experimental overexpression of ILP1 (Increased Level of Polyploidyl), an endoreduplication enhancer, increases glucosinolate production and compensation (from undercompensation to overcompensation) in a genotype of A. thaliana that typically suffers reduced fitness when damaged (Scholes & Paige (2014) Molecular Ecology 23:4862-4870; Mesa et al. (2017) Ecology 98: 2528-2537), demonstrating a causal relationship between the process of endoreduplication, fitness compensation (tolerance), and chemical defense.

Overall, these results translate into a positive relationship between plant tolerance and resistance (Mesa et al. (2017) Ecology 98; 2528-2537). Despite the molecular constraints that lead to a positive association between tolerance and resistance, the lack of a negative correlation does not preclude evidence of a trade-off given the costs in maintaining both strategies, as both strategies utilize carbon skeletons from a shared resource pool in the oxidative pentose phosphate pathway.

There is a need for reproducible, cost-effective, and sustainable methods for improving plant agronomic traits. Climate change is reducing the amount of arable land around the world. At the same time, the world population is increasing. A petrochemical-based agriculture that uses large amounts of fuel and organic pesticides to meet food demands is not sustainable because of climate change. Plant-based fuels must replace petrochemical fuels to reduce global warming, but cultivation of oil seed crops competes with much needed food production. As a consequence, there is a growing demand for methods and new plant cultivars capable of improving agronomic traits such as yield, drought tolerance, disease resistance, and more.

US 2022/0312710 A1 provides a method of improving agronomic traits of plants, including, but not limited to, yield, drought tolerance, and disease resistance by removing the plant's shoot apical meristem at an advantageous time in the growth cycle of the plant. For example, this application discloses removal of the shoot application meristem of soybean plants between vegetative stage 1 and 2 or vegetative stage 2 and 3, improves a number of agronomic traits including seed yield and vigor.

Further, US 2017/0067072 A1 describes transgenic plants that express ANAC055 polypeptide, which exhibit enhanced yield-related traits; U.S. Pat. No. 6,245,717 claims the use of the antiauxin 4-phenylbutyric acid to improve crop yield; and KR 100674115 B1 discloses a method for increasing yield of horseradish root in culture by removing the apical meristem of the plant. While removal of the shoot apical meristem has been suggested to increase plant yield of soybean plants (Toledo, et al. (2009) Acta Sci. Agron. 31(1):113-119; Parker (2016) Master's Thesis, “Agronomic Management of Soybean with Foliar Manganese and Apical Meristem Alterations,” University of Illinois at Urbana-Champaign; Sonderegger (2013) Master's Thesis, “High Yield Soybean Management: Planting Practices, Nutrient Supply, and Growth Modification,” University of Illinois at Urbana-Champaign; 2016 Regional Report, “Breaking Apical Dominance in Soybean,” Monsanto), apical meristem removal is suggested at or after vegetative growth state 2 and lead to decrease seed yields.

### SUMMARY OF THE INVENTION

This invention provides a method for improving one or more agronomic traits of a plant by selecting a plant variety that exhibits overcompensation and removing the apical meristem of plants of the plant variety (e.g., by physical removal by cutting or chemical removal) thereby improving one or more of the agronomic traits of the plants. In some aspects, the one or more agronomic traits comprise seed number, pod number, seed biomass, pod biomass, seed oil content, seed protein content, plant biomass, root biomass, leaf biomass, tuber biomass, fruit biomass, stem biomass, wood biomass, drought tolerance, or disease tolerance. In some aspects, the plants of the selected plant variety are modified to carry one or more mutations in one or more genes in a biosynthetic pathway for production of one or more chemical defense compounds (e.g., glucosinolates, terpenes, alkaloids, cyanogenic glycosides, or phenolics) such that the modified plants of the selected plant variety produce less of the chemical defense compounds compared a corresponding unmodified plant. In some aspects, the plant is soybean, corn, canola, rape, rice, potato, sunflower, flax, safflower, or camelina.

This invention also provides a method of improving one or more agronomic traits of a plant by inhibiting or reducing the amount of one or more chemical defense compounds produced by the plant (e.g., glucosinolates, terpenes, alkaloids, cyanogenic glycosides, or phenolics) to produce a modified plant and removing the apical meristem of the modified plant (e.g., by physical removal by cutting or chemical removal) to cause overcompensation or increase overcompensation of the modified plant compared to an unmodified plant thereby improving one or more agronomic traits of a plant. In some aspects, the one or more agronomic traits comprise seed number, pod number, seed biomass, pod biomass, seed oil content, seed protein content, plant biomass, root biomass, leaf biomass, tuber biomass, fruit biomass, stem biomass, wood biomass, drought tolerance, or disease tolerance. In some aspects, the modified plant carries one or more mutations in one or more genes in a biosynthetic pathway for production of one or more chemical defense compounds such that the modified plant produces less of the chemical defense compounds compared a corresponding unmodified plant. In some aspects, the plant is soybean, corn, canola, rape, rice, potato, sunflower, flax, safflower, or camelina.

The present invention also provides a method of producing one or more plant products from a modified plant that exhibits improvements in one or more agronomic traits by (a) growing a modified plant carrying one or more mutations in one or more genes in a biosynthetic pathway for production of one or more chemical defense compounds (e.g., glucosinolates, terpenes, alkaloids, cyanogenic glycosides, or phenolics) such that the modified plant produces less of the chemical defense compound compared the corresponding unmodified plant, (b) removing the apical meristem of the modified plant (e.g., by physical removal by cutting or chemical removal) to cause overcompensation or increased overcompensation of the modified plant compared to an unmodified plant, (c) growing the modified plant, and (d) harvesting one or more products produced by the modified plant. In some aspects, the one or more agronomic traits comprise seed number, pod number, seed biomass, pod biomass, seed oil content, seed protein content, plant biomass, root biomass, leaf biomass, tuber biomass, fruit biomass, stem biomass, wood biomass, drought tolerance, or disease tolerance. In some aspects, the plant is soybean, corn, canola, rape, rice, potato, sunflower, flax, safflower, or camelina. In other aspects, the one or more plant products comprise seeds, fruit, pods, tubers, roots, leaves, flowers, stems, fiber, and/or phytochemicals.

### BRIEF DESCRIPTION OF THE DRAWINGS

FIG. 1 shows regression of rosette diameter and total seeds of A. thaliana for both Col-0 and cyp79B2 cyp79B3 double mutant lines. Data show a significant positive relationship between the two variables.

FIG. 2 shows composite seed production for clipped and unclipped plants for Col-0 and cyp79B2 cyp79B3 double mutant lines. Shown are means±1 SE. Asterisks indicate significance at a familywise error rate of α=0.05 for clipped and unclipped plants.

FIG. 3 shows composite seed production for clipped and unclipped plants for Col-0 and cyp79B2 cyp79B3 double mutant lines adjusted for rosette size as a covariate. Shown are means±1 SE. Asterisks indicate significance at a familywise error rate of α=0.05 for clipped and unclipped plants.

FIG. 4 shows percent differences in composite seed yield and indole glucosinolate production comparing clipped and unclipped plants for 7 Arabidopsis thaliana genotypes. Genotypic responses range from undercompensation (negative percent difference values) to overcompensation (positive percent difference values). Asterisks indicate significance at a familywise error rate of α=0.05 between clipped and unclipped plants. (Figure taken from Mesa et al. (2017) Ecology 98: 2528-2537).

### DETAILED DESCRIPTION OF THE INVENTION

The following descriptions and examples illustrate embodiments of the present disclosure in detail. Although the present disclosure has been described in some details by way of illustration and example for purposes of clarity and understanding, it will be apparent that certain changes and modifications can be practiced within the scope of the appended claims.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment. It is to be understood that the present disclosure is not limited to the particular aspects described herein and as such can vary. Those of skill in the art will recognize that there can be variations and modifications of the present disclosure, which can be encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated cases, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular aspects only and is not intended to be limiting.

In this application, the use of the singular includes the plural unless specifically stated otherwise. It must be noted that, as used in the specification, the singular forms “a”, “an”, and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C”. The term “or” can be used conjunctively or disjunctively unless the context specifically refers to a disjunctive use.

Furthermore, the use of the term “including” as well as other forms, such as “include”, “includes” and “included”, is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed. The term “under suitable condition” or “under suitable reaction condition” refers to any environment that permits a desired reaction to take place.

The term “isolated” refers to a state where it is partially, substantially, or completely free of the materials with which it is associated in nature. By partially or substantially free is meant at least 0.1%, at least 0.5%, at least 1%, at least 5%, at least 10%, at least 20%, at least 30%, at least 40%, at least 50%, at least 60%, at least 70%, at least 80%, at least 90%, at least 99%, or 100% free of the materials with which it is associated in nature, inclusive of all values falling in between these percentages. Accordingly, as used herein, an “isolated enzyme” refers to an enzyme that has been partially, substantially, or completely has been separated from its biological source (e.g., microbial organism, yeast, bacteria, etc.). The isolated enzyme may or may not be combined in a formulation with other ingredient for application disclosed herein. An isolated enzyme may or may not be purified (e.g., free from other environmental contaminants, microbial secretes, or deactivated organisms, etc.), but it is separated from the source organisms, or the source organisms have been deactivated. Thus, for purpose of the present disclosure, isolated enzymes include not only purified enzymes, but also enzymes and mixtures of enzymes present in the source organism culture medium or extract.

It is intended that every maximum numerical limitation given throughout this specification include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Some plants can mitigate the damage caused by animal herbivores by increasing their fitness when damaged. This phenomenon is termed overcompensation. It has been demonstrated that Arabidopsis thaliana ecotype Columbia-4 employs endoreduplication, the replication of the genome without mitosis, following removal of the shoot apical meristem and that it overcompensates for seed yield (Scholes & Paige (2014) Molecular Ecology 23:4862-4870). A related genotype Landsberg erecta does not increase its endoreduplication following removal of the shoot apical meristem and suffers reduced seed yield following removal of the shoot apical meristem. By analyzing the progeny of a cross of Columbia-4 x Landsberg erecta it has been found that endoreduplication is directly involved in compensatory performance. Another A. thaliana variety, Columbia-0, is an undercompensator, that is, it does not increase seed yield after removal of the shoot apical meristem compared to control plants with an intact shoot apical meristem. In fact, following removal of the Columbia-0 shoot apical meristem, the plant produces fewer seeds than control plants with an intact apical meristem. After removal of the shoot apical meristem, Columbia-0 does not endoreduplicate.

There is a need to improve plant agronomic traits. There is a need for more plant varieties that are capable of overcompensating when the apical meristem is removed, or the growth of the apical meristem is inhibited. There is a need for more plant varieties that exhibit improved overcompensation when the apical meristem is removed, or the growth of the apical meristem is inhibited. Plants that overcompensate exhibit improved agronomic traits including, but not limited to, one or more of increased seed yield, improved drought tolerance, and improved disease tolerance compared to control plants with intact and functional apical meristems. With the limited amount of high quality arable land that is available for row crop production in regions having suitable climate, any method that would improve the vigor and yield of agronomic plants in general, and in particular, for legumes, such as soybeans, would provide a significant advantage.

Accordingly, the present invention provides methods for improving one or more agronomic traits (e.g., seed yield, seed oil content, seed protein content, plant biomass, drought tolerance, and disease tolerance) of plants by selecting and/or generating (e.g., by breeding or genetic modification) a plant variety that exhibits overcompensation; and removing the apical meristem of plants of the plant variety (e.g., by chemical or physical means) thereby improving one or more of the agronomic traits of the plants. In some aspects, the selected plants are modified to carry one or more mutations in one or more genes in a biosynthetic pathway for production of one or more chemical defense compounds such that the modified plant produces less of the chemical defense compound compared a corresponding unmodified plant. The methods disclosed herein provide for control of plant overcompensation. The methods disclosed herein provide for converting a plant that does not overcompensate into a plant that overcompensates. The methods disclosed herein provide for increasing the ability of a plant to overcompensate. Once the plant is capable of overcompensating then, removal or inhibition of growth of the apical meristem, referred to herein as clipping, at an appropriate time in the plant growth cycles improves the plant's agronomic traits, including, but not limited to yield, vigor, drought tolerance, and disease resistance.

The term “plant” is to be understood as a plant of economic importance and/or cultivated plant. A plant is preferably selected from an agricultural, silvicultural, and horticultural (including ornamental) plant. The term “plant” as used herein includes all parts of a plant such as germinating seeds, emerging seedlings, herbaceous vegetation as well as established woody plants including all belowground portions (such as the roots) and aboveground portions. Generally, the term “plant” also includes a plant that has been modified by breeding, mutagenesis, or genetic engineering. Genetically modified plants are plants, which genetic material has been modified by the use of recombinant DNA techniques. The use of recombinant DNA techniques makes modifications possible that cannot readily be obtained by cross breeding under natural circumstances, mutations, or natural recombination.

The term “plant selection” or “selecting a plant” means a plant with desirable characteristics chosen over others in a plant breeding program. The plant may be selected based on traits such as disease resistance, drought tolerance, yield, flavor, or any other characteristic that a breeder might be interested in. The selected plant is then used in further breeding efforts to perpetuate and increase the prevalence of these traits in future generations. In some aspects, a plant or plant variety is selected for exhibiting overcompensation.

The term “plant variety” means refers to a specific type of a plant grouping within a plant species. Each variety has a unique combination of characteristics that distinguish it from other varieties within the same species. These could include features such as color, size, resistance to diseases, growth habit, flavor, yield, and so on. Varieties may arise naturally or can be specifically developed through breeding programs.

The term “plant inbred” means a plant that is the result of inbreeding, a process which involves the mating of closely related individuals within the same species. In plant breeding, this is often done to maintain certain desirable traits within a population or generate genetically more uniform parents for production of plant hybrids. Prolonged inbreeding can lead to reduced genetic diversity and can bring out harmful recessive traits, a phenomenon known as inbreeding depression. However, crossing two inbred plants may result in a hybrid that exhibits increased vigor, better yield, and other desirable characteristics.

The term “plant hybrid” means the plant produced from the cross of two different parent plants, typically from the same species but different inbred, varieties, or cultivars. The aim is to produce offspring that possess the best characteristics of both parents. This could include traits such as disease resistance from one parent, and high yield from another. Hybrid plants often exhibit what's called hybrid vigor or heterosis, where they perform better than both of their parents in certain traits.

Agricultural plants of use in the methods of this invention include, for example, cereals, for example wheat, rye, barley, triticale, oats or rice; beet, for example sugar beet or fodder beet; fruits, such as pomes, stone fruits or soft fruits, for example apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as broccoli, spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; natural rubber plants or ornamental and forestry plants, such as flowers, shrubs, broad-leaved trees or evergreens, for example conifers; and on the plant propagation material, such as seeds, and the crop material of these plants. In some aspects, the plant is a leguminous plant, such as lentil, pea, alfalfa, or soybean. In some aspects, the plant is selected from the group consisting of soybean, corn, canola, rape, rice, potato, sunflower, flax, safflower, and camelina.

Plants possess multiple types of defenses against herbivores, pathogens, and pests. The pathogens, herbivores, and pests that feed on plants include a diverse array living organisms including insects, nematodes, larger animals, bacteria, fungi, and viruses. One type of defense involves production of chemical compounds that prevent or deter infestation. These chemical defense compounds can be produced constitutively, or their production can be induced by infestation. Another plant strategy is production of structural features that deter or prevent infestation. These features can include thorns, spines, and trichomes. Trichomes frequently contain chemicals that are toxic to, or deter, infestation.

Yet another defense strategy is regrowth or fitness strategies that are referred to as tolerance. One such regrowth or tolerance strategy is overcompensation. Overcompensation occurs when the apical meristem of the plant is removed, or the growth of the apical meristem is inhibited. As a result, endoreduplication occurs in which the genome of plant cells replicates in the absence of mitosis leading to cellular polyploidy.

These strategies of chemical defense, structural defense, and overcompensation typically occur concurrently within the same plant. Multiple defense mechanisms may be costly to a plant, as investment in chemical defense may reduce resources available for overcompensation. Likewise, an investment in overcompensation may reduce resources available for chemical defense. The biochemical pathways that support overcompensation and may also support chemical resistance. If so, it may be possible to divert carbon skeletons from the biochemical pathways that support chemical resistance into the pathways that support overcompensation, thereby establishing overcompensation in a plant that did not exhibit this trait, or increase the expression of overcompensation in a plant that does exhibit this trait.

The gene glucose-6-phosphate-1-dehydrogenase (G6PD1, At5g35790.1) appears to play a role in controlling the overcompensation response in Arabidopsis thaliana following the removal of the plant's primary inflorescence. Overcompensation in response to herbivory in Arabidopsis thaliana: the role of glucose-6-phosphate dehydrogenase and the oxidative pentose-phosphate pathway. G6PD1 is the central regulatory enzyme in the oxidative pentose phosphate (OPP) pathway that plays a key role in plant metabolism generating NADPH and a variety of metabolic intermediates for biosynthetic processes including resistance to oxidative damage, the production of plant secondary defensive compounds, such as glucosinolates and the production of ribo- and deoxyribonucleotides.

Thus, the term “capable of overcompensation,” “overcompensation” or “overcompensator” refers to a plant that exhibits one or more improved agronomic traits, including, but not limited to, yield, seed production, fruit production, biomass, drought tolerance, disease resistance, seed oil content, or vigor, after inhibition of growth or removal of the plant's apical meristem, when compared to a genetically similar or identical plant that has not had its apical meristem removed or the growth of its apical meristem inhibited.

The terms “incapable of overcompensation” or “undercompensator” refers to a plant that does not exhibit, or does not exhibit a significant increase in, one or more improved agronomic traits, including, but not limited to, increased yield, increased drought tolerance, increase disease resistance, or increase vigor, after inhibition of growth or removal of the plant's apical meristem.

In accordance with one or more aspects of this invention, one or more agronomic traits of a plant are improved. In some aspects, the agronomic trait is seed yield, seed oil content, seed protein content, plant biomass, drought tolerance, and/or disease tolerance.

As used herein, the term “yield” generally refers to a measurable product of economic value that is produced by the plant such as grains, fruits in the proper sense, vegetables, nuts, grains, seeds, wood (e.g., in the case of silviculture plants) or even flowers (e.g., in the case of gardening plants, ornamentals). The plant products may in addition be further used and/or processed after harvesting. According to the present invention, “increased yield” of a plant, in particular of an agricultural, silvicultural and/or ornamental plant means that the yield of a product of the respective plant is increased by a measurable amount over the yield of the same product of the plant produced under the same conditions, but without removing the shoot apical meristem of the plant. Increased yield can be characterized, among others, by increased plant weight, increased plant height, increased above- and/or below-ground biomass such as higher fresh and/or dry weight, higher grain yield, more tillers, larger leaves, increased shoot growth, increased seed number, increased seed weight, increased seed protein content, increased seed oil content, increased starch content and/or increased pigment content. In some aspects, yield is characterized by increased seed yield, seed oil content, seed protein content, and/or plant biomass.

Another indicator for the condition of the plant is the “plant vigor.” Plant vigor becomes manifest in several aspects such as the general visual appearance. Improved plant vigor can be characterized, among others, by improved vitality of the plant, improved plant growth, improved plant development, improved visual appearance, improved plant stand (less plant verse/lodging), improved emergence, enhanced root growth and/or more developed root system, enhanced nodulation, in particular rhizobial nodulation, bigger leaf blade, increased plant size, increased plant weight, increased plant height, increased tiller number, increased shoot growth, increased root growth (extensive root system), increased size of root mass (extensive root system), increased yield when grown on poor soils or unfavorable climate, enhanced photosynthetic activity, change of color (e.g., enhanced pigment content), earlier flowering, earlier fruiting, earlier and improved germination, earlier (advanced) grain maturity, improved abiotic and/or biotic stress tolerance, less non-productive tillers, less dead basal leaves, less input needed (such as fertilizers or water), greener leaves and increased green leaf area, complete maturation under shortened vegetation periods, less fertilizers needed, less seeds needed, easier harvesting, faster and more uniform ripening, longer shelf-life, longer panicles, delay of senescence, stronger and/or more productive tillers, better extractability of ingredients, improved quality of seeds (for being seeded in the following seasons for seed production), reduced production of ethylene and/or the inhibition of its reception by the plant, spindliness of leaves, and/or increased number of ears/m². In some aspects, the one or more agronomic traits of the plant is improved abiotic tolerance such as improved drought tolerance and/or improved biotic stress tolerance such as improved disease tolerance.

The improvement or increase in yield and/or vigor according to the present invention particularly means that the improvement of any one or several or all of the above-mentioned plant characteristics are improved compared to a plant produced under the same conditions, but without removing the shoot apical meristem of the plant (i.e., an unmodified plant). Yield and yield increase (in comparison to a wild-type, unmodified plant) can be measured in a number of ways. It is understood that a skilled person will be able to apply the correct meaning in view of the particular aspects, the particular crop concerned, and the specific purpose or application concerned.

In one aspect of the present invention, yield and/or vigor is increased by at least 5% to 45% (or any range derivable therein). In one embodiment, the yield and/or vigor is increased by least 10%. According to another embodiment of the present invention, the yield and/or vigor is increased by least 20%. According to another embodiment of the present invention, the yield and/or vigor is increased by least 30%. According to another embodiment of the present invention, the yield and/or vigor is increased by least 40%. According to another embodiment of the present invention, the yield and/or vigor is increased by least 42%. By way of example, if untreated soybeans yielded 6200 bushels of seeds per 100 acres, and if soybeans that received the subject treatment yielded 8500 bushels of seeds per 100 acres under the same growing conditions, then the yield of soybeans would be said to have been increased by ((8500−6200)/6200)×100=37%.

In certain aspects, improved or increased “yield” refers to one or more yield parameters selected from the group of biomass yield, dry biomass yield, aerial dry biomass yield, underground dry biomass yield, fresh-weight biomass yield, aerial fresh-weight biomass yield, underground fresh-weight biomass yield, and/or preferably enhanced yield of seeds (either dry or fresh-weight, or both). In one embodiment, an increase in yield refers to increased harvestable yield, biomass yield and/or an increased seed yield. Biomass yield may be calculated on a per plant basis or in relation to a specific area (e.g., biomass yield per acre/square meter/or the like).

The “harvestable yield” of a plant can depend on the specific plant/crop of interest as well as its intended application (such as food production, feed production, processed food production, biofuel, biogas, or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like.

“Biomass yield” can refer to, e.g., dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. In some aspects, the biomass is seed biomass, pod biomass, plant (total above-ground portion of the plant) biomass, root biomass, leaf biomass, tuber biomass, fruit biomass, stem biomass, and/or wood biomass.

“Seed yield” can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seed weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g., at 15.5 percent moisture.

In some aspects, an increase in yield is conferred by an increase of the intrinsic yield capacity of a plant and can be, for example, manifested by improving the specific (intrinsic) seed yield (e.g., in terms of increased seed/grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigor/early vigor, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

In one aspect, an increase in yield is conferred by an improvement or increase of stress tolerance of a plant and can be for example manifested by improving or increasing a plant's tolerance against stress, particularly abiotic stress. In the present application, abiotic stress refers generally to abiotic environmental conditions a plant is typically confronted with, including conditions which are typically referred to as “abiotic stress” conditions including, but not limited to, drought (tolerance to drought may be achieved as a result of improved water use efficiency), heat, low temperatures and cold conditions (such as freezing and chilling conditions), salinity, osmotic stress, shade, high plant density, oxidative stress, and the like.

In another aspect, an increase in yield is conferred by increasing the nutrient use efficiency of a plant, e.g., by improving the use efficiency of nutrients including, but not limited to, phosphorus, potassium, and nitrogen. For example, there is a need for plants that are capable to use nitrogen more efficiently so that less nitrogen is required for growth and therefore resulting in the improved level of yield under nitrogen deficiency conditions. Further, higher yields may be obtained with current or standard levels of nitrogen use.

In some aspects, improved or increased “vigor” refers to one or more parameters selected from the group of tolerance to environmental stress and/or biotic stress, and/or seed yield, seed quality, and/or biomass production.

The methods disclosed herein contemplate removing or inhibiting the growth of one or more plant apical meristems at a time, or in a time period, advantageous for improving one or more agronomic traits of the plant. In some aspects, one or more plant apical meristems are removed by clipping. In other aspects, the growth of one or more plant apical meristems is inhibited or removed by, e.g., chemical means.

The term “meristem” means a region of cells capable of division and growth in plants. Meristematic cells are typically small and nearly spherical. They have a dense cytoplasm and relatively few small vacuoles. Some of these meristematic cells maintain the meristem as a continuing sour of new cells and may undergo cell division (mitosis) many times before differentiating into specific cells required for that region of the plant body.

The term “apical meristem” means the meristem and the shoot or root tips of the plant. The apical meristem gives rise to the primary plant body and is responsible for the extension of the roots and shoots. The shoot apical meristem is a center of potentially indefinite growth and development producing the leaves as well as a bud in the axis of most leaves that has the potential to grow out as a branch. The shoot apical meristem generates all the aerial organs including the floral meristems. The plant hormone auxin is produced in the shoot apical meristem. Among the many roles of auxin in plant development, it inhibits the production of lateral branches.

The term “lateral meristems” means the meristem in the vascular and cork cambia. Lateral meristems are known as secondary meristems because they are responsible for secondary growth or increase in stem girth and thickness.

The term “intercalary meristem” means the meristem at the internodes or stem regions between the places at which leaves attach.

The term “endoreduplication or endoreplication” means the replication of the nuclear genome in the absence of mitosis (cell division). Endoreduplication results in elevated nuclear gene content and polyploidy.

The term “clipping” means removal or inhibition of the growth of the shoot apical meristem of a plant by any means of mechanical trimming or chemical treatment. Mechanical trimming is accomplished by mowing, pruning by hand, or any other method of severing the apical meristem in whole, or in part, from the plant. Chemical treatment is accomplished using any chemical composition that inhibits the growth, or kills a significant number of cells in the shoot apical meristem, without significantly damaging growth of other parts of the plant.

As is conventional in the art, the shoot apical meristem is the region in the growing shoot containing meristematic cells. The shoot apical meristem contains multipotent stem cells and produces primordia that develop into all the above ground organs of a plant. In accordance with the methods herein, the shoot apical meristem is removed at a period between vegetative growth stage 1 (V1) and vegetative growth stage 2 (V2); or a period between vegetative growth stage 2 (V2) and vegetative growth stage 3 (V3). Preferably, removal of the shoot apical meristem results in minimal or no removal of adjacent V1 tissue if clipping between V1 and V2, or minimal or no removal of adjacent V2 tissue if clipping between V2 and V3. Shoot apical meristem removal can be by physical/mechanical (e.g., cutting, crushing, or clipping) or chemical (e.g., application of lactofen) means. When carried out by physical/mechanical means, preferably a cross-sectional cut of the plant is carried out just below the shoot apical meristem. Further, it is posited that physical/mechanical removal of the shoot apical meristem can be performed manually or by automation. For example, a robot such as WP5 (Wageningen University, The Netherlands), Wall-Ye (Macon, France), or strawberry harvester (Agrobot; La Palma del Condado, Huelva, Spain) could be adapted to autonomously remove shoot apical meristems from crop plants.

It is understood that a skilled person will be able to determine/identify the V1, V2 and V3 stages of a particular crop concerned. For example, V1 of determinate and indeterminate dry bean such as soybean is when the first fully developed trifoliolate at the third node appears, e.g., at approximately 10-20 days after seeding; V2 is when the second trifoliolate (count when leaf edges no longer touch) appears at approximately 19-25 days after seeding; and V3 is when the third trifoliate appears after 25-32 days after seeding. Similar to dicots, V1 of a monocot such as corn is when the first round-tipped leaf on first collar appears, and nodal roots elongate. By V2, the monocot may be 2 to 4 inches tall and rely on energy in the seed. V3 begins 2 to 4 weeks after VE (emergence), and the plant switches from kernel reserves to photosynthesis and nodal roots begin to take over. Notably, in corn, a plant with 3 collars is considered V3, however, there may be 5 to 6 leaves showing on the plant. Given that growth stages can overlap, a crop of plants is in a particular growth stage when 50% or more of the plants of the crop are in or beyond that stage. Moreover, if senescence of the lower leaves has occurred, leaf scars (excluding those where the cotyledons were attached) are counted to determine the proper stage. In accordance with the present method, it is preferable that shoot apical meristem removal occurs within the 3-to-10-day window between stages V1 and V2, or V2 and V3, or more preferably within a 5-day window between stages V1 and V2 or V2 and V3.

In some aspects, the removing step includes a cross-sectional cut below the shoot apical meristem or applying a chemical to the shoot apical meristem of a plant. In some aspects, removing the plant's shoot apical meristem improves one or more plant agronomic traits including, but not limited to, increasing seed yield, seed weight, aboveground biomass, belowground biomass, tuber size, tuber number, or secondary metabolite production. Per hectare yield can be increased by 1, 5, 10, 15, 20, 25, 30, 35, 40, 45, 50, 60, 70, or more percent through the removal, or inhibition of growth, of the plant apical meristem. Depending on the plant variety, geographic region, or agronomic conditions, the apical meristems of the plants can be removed early or later in plant growth.

Following the removal of the plant apical meristem, phenotypically plastic increases in ploidy level via endoreduplication lead to rapid regrowth and an increase in fitness, explaining, in part, the phenomenon of overcompensation in plants. Endoreduplication is the replication of the genome without mitosis, which leads to endopolyploidy, an increase in cellular chromosome number. Removal of the apical meristem by herbivores eliminates production of the plant hormone auxin, leading to a rapid break in dormancy of axillary buds and subsequent stem elongation. High levels of auxin are also known to repress the endocycle, and by contrast, lower levels of auxin trigger an exit from mitotic cycles and an entry into endocycles. Insect leaf-feeding also can trigger endoreduplication by the upregulation of jasmonic acid, which also lowers auxin production and can lead to overcompensation in some ecotypes of Arabidopsis. Thus, there is a direct link between endoreduplication and plant damage.

Increasing chromosome number through endoreduplication and therefore gene copy number provides a means of increasing expression of vital genes or genetic pathways that promote rapid regrowth rates following removal, or inhibition of growth, of the apical meristem. G6PD1 feeds compounds into the OPP pathway for nucleotide biosynthesis, by the provision of ribose-5-phosphate, necessary for the significant increase in chromosome number via endoreduplication. The increase in DNA content then feeds back positively on pathways involved in metabolism (e.g., G6PD1) and chemical defense. Endoreduplication leads to increased gene copy number and therefore increased gene expression. The chemical defense compounds implicated in this process include, but are not limited to, glucosinolates, terpenes, alkaloids, among others.

The present invention also provides methods of establishing overcompensation in a plant that does not exhibit this trait, or increasing overcompensation in a plant that already exhibits the trait, by diverting carbon skeletons from a shared resource pool in the oxidative pentose phosphate pathway, away from chemical defense compounds, thereby increasing the plant's ability to support biochemical pathways associated with overcompensation. The present invention is exemplified, but is not limited to, an example of an undercompensating wild-type Columbia-C Arabidopsis thaliana ecotype that could be made capable of overcompensation. This was accomplished with mutations in one or more genes coding for two cytochrome P450s. The two cytochrome P450 genes code for enzymes involved in the biosynthetic production of the chemical defense compounds indole glucosinolates. The wild type versions of the mutated P450 genes code for enzymes that convert tryptophan to indole-3-acetaldoxime (IAOx). IAOx is then further used in the production of indole glucosinolates. A mutation in the A. thaliana Columbia-0 ecotype indole glucosinolate pathway leads to a decrease in the indole glucosinolate chemical defense compounds and an increase in the ability of the A. thaliana mutant plant to overcompensate when its apical meristem is removed.

Thus, in some aspects, the invention provides a method of improving one or more agronomic traits of a plant by inhibiting or reducing the amount of one or more chemical defense compounds produced by the plant to produce a modified plant; and removing the apical meristem of the modified plant, e.g., at a time in the modified plant's growth cycle that causes overcompensation or increases overcompensation compared to the unmodified plant, and optionally growing the modified plant that exhibits one or more improved agronomic traits.

In another aspect, the invention provides a method for producing one or more plant products from a modified plant that exhibits improvements in one or more agronomic traits, by (a) growing a modified plant that carries one or more mutations in one or more genes in a biosynthetic pathway for production of one or more chemical defense compounds such that the modified plant produces less of the chemical defense compound compared a corresponding unmodified plant, (b) removing (e.g., physical removal by means of mechanical clipping at a designated stage in the development of the plant) or inhibiting the growth of (e.g., by means of chemical treatment at a designated stage in the development of the plant) the apical meristem of the modified plant at a time in the modified plant's growth cycle that causes overcompensation, or increased overcompensation, compared to the unmodified plant, (c) growing the modified plant, and (d) harvesting one or more products produced by the modified plant.

The term “plant products” or “products produced by a plant” refers to one or more portions of a plant that is obtained at some time during the growing cycle. In some aspects, the plant products are commercially relevant portions of the plant and include, e.g., a tuber, seed, seed pod, fruit, leaf, stem, root, or one or more phytochemicals or processed portions of the plant. A plant product may be any of one or more natural plant products such as starch, oil, protein, carbohydrates, terpenes, or more.

A “modified plant” is a plant that carries one or more mutations in one or more genes in a biosynthetic pathway for production of one or more chemical defense compounds such that the modified plant produces less of the chemical defense compound compared a corresponding unmodified plant. The one or more mutations in one or more genes in a biosynthetic pathway for production of one or more chemical defense compounds may be generated by mutagenesis or recombinant means.

Mutagenesis, the process of inducing genetic mutations, has been an integral part of plant science for many decades. It allows scientists to understand gene functions, create genetic diversity, and enhance crop traits such as yield, disease resistance, and environmental resilience. Methods of mutagenesis are well known to the skilled artisan.

Physical mutagenesis involves the use of physical agents to induce mutations. These methods cause changes in the DNA structure, leading to mutations. Radiation mutagenesis is one of the oldest techniques used in plant mutagenesis. It involves the use of ionizing radiation such as X-rays, gamma rays, and fast neutrons, to induce DNA damage, often resulting in deletions, inversions, translocations, or duplications. The resulting population of plants is screened for mutants of interest. This technique has been instrumental in developing numerous crop varieties.

Chemical mutagenesis employs various chemicals that can modify or damage DNA, causing mutations. These are chemicals that can substitute for normal nucleotides during DNA replication. Examples include 5-bromouracil and 2-aminopurine, which can pair erroneously, causing point mutations. Alkylating agents such as ethyl methane sulfonate (EMS) and N-nitroso-N-methylurea (NMU), can donate alkyl groups to DNA bases, leading to miscoding and subsequent point mutations. Deaminating agents can remove amino groups from bases, leading to altered base-pairing properties. An example is nitrous acid, which can cause transitions or transversions. Following chemical mutagenesis, the resulting population of plants is screened for mutants of interest.

Insertional mutagenesis involves inserting a piece of DNA, such as a transposon or T-DNA, into the plant genome, disrupting gene function at the insertion site. Transposons, or “jumping genes”, can move within the genome, creating mutations wherever they insert. This method allows for “tagging” of genes, enabling their subsequent identification and isolation. T-DNA mutagenesis uses Agrobacterium tumefaciens, a bacterium that naturally transfers its T-DNA into the plant genome. T-DNA is manipulated, allowing for the insertion of desired sequences, gene disruption, or gene tagging. See, for example, Azpiroz-Leehan & Feldmann (1997) Trends Genetics 13(4):152-156.

Targeted mutagenesis allows for the creation of precise mutations at specific sites in the genome. Site-directed mutagenesis is typically performed in vitro and involves the use of modified oligonucleotides to introduce specific mutations during DNA replication. See, for example, Smith (1985) Ann. Rev. Genetics 19(1):423-462. Genome editing through the use of CRISPR/Cas9, and other related systems, allows for precise edits in the plant genome, creating specific insertions, deletions, or substitutions. See, for example, Hsu et al. (2014) Cell 157(6):1262-1278. While technically not mutagenesis, as the process is directed and specific, genome editing techniques are the logical successors to traditional mutagenesis methods due to their high precision and efficiency.

Plants have evolved an array of defense mechanisms including synthesis of an impressive array of chemical compounds as part of their defense mechanism. These chemical defense compounds are the products of intricate biosynthetic pathways, allowing plants to effectively combat pests and pathogens. In the present invention, it has been found that the defense mechanisms of overcompensation can be established in a plant that does not normally express the trait, or expression of overcompensation can be increased when the production of chemical defense chemicals is inhibited or reduced. The biosynthetic pathways for production of chemical defense compounds, and the genes that code for the enzymes in these pathways, are well known. It is well within the skill in the art of plant molecular biology and plant breeding, to introduce mutations into specific genes in these pathways using CRISPR/Cas9, or similar techniques, to inhibit or reduce expression of one or more chemical defense compounds.

In some aspects, the chemical defense compound is a glucosinolate. Glucosinolates are sulfur-containing compounds found in the Brassicaceae family, which includes broccoli, cabbage, and mustard. When tissue damage occurs (for instance, when an insect starts eating the plant), glucosinolates are hydrolyzed by the enzyme myrosinase, producing compounds like isothiocyanates and nitriles, which are toxic to many organisms.

Glucosinolate biosynthesis involves a complex pathway starting from amino acids. In brief, the amino acid is first chain-elongated, then the core glucosinolate structure is formed by sequential oxidation, decarboxylation, and sulfation. Myrosinase and glucosinolates are stored separately in the plant cells, effectively forming a two-component system that is activated upon tissue damage.

Glucosinolates constitute a large and diverse group of chemical defense compounds found in a wide range of plants including Brassicales (including A. thaliana, broccoli, cabbage). Glucosinolates (mustard oil glucosides) are nitrogen and sulfur rich natural plant secondary products that consist of a sulfonated oxime and a β-thioglucose moiety, but differ in side-chain structures. There have been ˜40 glucosinolates found in Arabidopsis. Indole and aliphatic glucosinolates constitute most of the diversity of glucosinolates in A. thaliana. Indole glucosinolates are composed of four individual compounds: glucobrassicin, 4-methoxy-glucobrassicin, neoglucobrassicin and 4-hydroxyglucobrassicin.

Many studies have shown that glucosinolate breakdown products deter generalist and specialist herbivores, pests, and pathogens. With damage of plant cell integrity, glucosinolates stored in the vacuole are mixed with the enzyme myrosinase, a β-thioglucosidase that is separated in scattered specialist cells known as myrosin cells. Myrosinase cleaves the β-glucose moiety from glucosinolates, leading to a variety of toxic breakdown products, such as bioactive nitriles, epithionitriles and isothiocyanates based on reaction conditions and protein factors such as epithiospecifier protein. In A. thaliana, indole glucosinolate synthesis involves a catalyzed conversion of tryptophan to indole-3-acetaldoxime (IAOx) carried out by two cytochrome P450s, cyp79B2 and cyp79B3. See, Hull et al. (2000) Proceedings of the National Academy of Sciences 97:2379-2384. IAOx is further catalyzed through four subsequent reactions to form glucobrassicin, the most abundant indole glucosinolate found in A. thaliana. See, Bender & Celenza (2009) Phytochemistry Reviews 8:25-37. Glucobrassicin can then be further modified to the other three indole glucosinolates found in A. thaliana with modified indole rings. See, Pfalz et al. (2009) The Plant Cell 21:985-999.

In some aspects, the chemical defense compound is an alkaloid. Alkaloids are a broad group of compounds that are often bitter tasting and toxic. These nitrogen-containing compounds deter herbivores and pathogens due to their wide range of biochemical effects. The biosynthesis of alkaloids usually involves amino acids as primary precursors. For instance, the well-known alkaloid nicotine, found in tobacco plants, starts with the decarboxylation of putrescine, a product of the amino acid ornithine or arginine. This creates N-methylputrescine which is further metabolized into nicotine via a series of reactions.

In some aspects, the chemical defense compound is a terpenoid or isoprenoid. Terpenoids represent the largest and most structurally diverse group of plant metabolites. They are made up of repeating units of a 5-carbon molecule isoprene. Terpenoids can act as repellents, toxins, or antifeedants to pests. The biosynthesis of terpenoids takes place through the mevalonate pathway or the methylerythritol phosphate pathway. An example is the biosynthesis of artemisinin, a sesquiterpene lactone with antimalarial properties found in Artemisia annua. It's synthesized through the mevalonate pathway, where farnesyl diphosphate (FDP), a common precursor for sesquiterpenes, is cyclized to amorpha-4,11-diene by the enzyme amorpha-4,11-diene synthase. Subsequent oxidations and rearrangements lead to artemisinin.

In some aspects, the chemical defense compound is a phenolic compound. Phenolic compounds include simple phenols, phenolic acids, quinones, flavonoids, tannins, and lignins. They serve multiple roles including UV protection, structural support, and of course, defense against pests and pathogens. Phenolics are generally synthesized through the shikimate and phenylpropanoid pathways. For example, flavonoids begin with phenylalanine, which is deaminated to cinnamic acid by the enzyme phenylalanine ammonia-lyase (PAL), This is then converted through a series of enzymatic steps to yield flavonoid compounds.

In some aspects, the chemical defense compound is a cyanogenic glycoside. Cyanogenic glycosides are compounds that can be used by plants as a source of hydrogen cyanide, a potent toxin, which is released when the plant tissues are damaged. These compounds play a crucial role in plant defense against herbivores. The biosynthesis of cyanogenic glycosides also initiates from amino acids. For instance, in the production of the cyanogenic glycoside linamarin, the amino acid valine is converted into 2-oxoisovalerate, which is then converted into the cyanohydrin, and finally glycosylated to form linamarin.

The following non-limiting examples are provided to further illustrate the present invention.

### Example 1: Arabidopsis thaliana Col-0 and Col-0 Double Mutant cyp79B2 cyp79B3

Arabidopsis thaliana, mouse-ear cress, is a small, mostly selfing plant in the Brassicaceae family. While native to Europe, A. thaliana has a wide geographical range spanning Eurasia, North Africa, and North America. Typically, A. thaliana is found as a winter annual where seeds of A. thaliana germinate in the fall after passing the summer in a dormant state and grow into an overwintering rosette, and following stem elongation in the spring, produce flowers that develop into seed pods known as siliques. A. thaliana is fed upon by a variety of species including flea beetles, aphids, leaf miners, caterpillars, deer, and rabbits. A. thaliana thus frequently experiences leaf and apical meristem damage and has a suite of defense strategies such as trichomes, proteinase inhibitors and glucosinolates that deter and inhibit feeding by herbivores, pests, and pathogens. A. thaliana also has a suite of tolerance strategies ranging from undercompensation to overcompensation. (Siddappaji et al. (2013) Genetics 195:589-598).

Glucosinolates constitute a large and diverse group of defensive secondary metabolites characteristic of the order Brassicales, which includes A. thaliana. Glucosinolates (mustard oil glucosides) are nitrogen- and sulfur-rich natural plant secondary products that are composed of a sulfonated oxime and a β-thioglucose moiety, but differ in side chain structures. Out of the 120 glucosinolates identified, most are classified into three subgroups based on the biosynthetic amino acid precursor, those subgroups being indole, aliphatic and benzenic. Indole and aliphatic glucosinolates constitute most of the diversity of glucosinolates in A. thaliana. Indole glucosinolates are composed of four individual compounds: glucobrassicin, 4-methoxy-glucobrassicin, neoglucobrassicin and 4-hydroxyglucobrassicin.

Many studies have shown that glucosinolate breakdown products deter generalist and specialist herbivores on A. thaliana. Upon herbivory, glucosinolates stored in the vacuole are mixed with the enzyme myrosinase (known as the “mustard bomb”), a β-thioglucosidase that is separated in scattered specialist cells known as myrosin cells. Myrosinase cleaves the β-glucose moiety from glucosinolates, leading to a variety of toxic breakdown products, such as bioactive nitriles, epithionitriles and isothiocyanates based on reaction conditions and protein factors such as epithiospecifier protein.

In A. thaliana, indole glucosinolate synthesis involves a catalyzed conversion of tryptophan to indole-3-acetaldoxime (IAOx) carried out by two cytochrome P450s, cyp79B2 and cyp79B3. IAOx is further catalyzed through four subsequent reactions to form glucobrassicin, the most abundant indole glucosinolate found in A. thaliana. Glucobrassicin can then be further modified to the other three indole glucosinolates found in A. thaliana with modified indole rings.

Costs of Resistance. To assess costs between plant tolerance and resistance, 100 seeds of Col-0 and 100 seeds of cyp79B2 cyp79B3 double mutant lines were planted and grown. While single cyp mutant lines show little deficiency in the ability to produce indole glucosinolates, double mutants are completely devoid of any indole glucosinolates. Of particular note, wild-type Col-0 on average produces a high concentration of indole glucosinolates when compared to other A. thaliana accessions.

The cyp79B2 cyp79B3 double mutant was uncovered from T-DNA insertion lines of cyp79B2 and cyp79B3 in the Col-0 background, identified from the Salk Institute collection of T-DNA insertion lines by PCR. Primers 79B2-5P (5′-TGGACAAGTATCATGACCCAATCATCCACG-3′; SEQ ID NO:1) and LB (5′-GGCAATCAGCTGTTGCCCGTCTCACTGGTG-3′; SEQ ID NO:2) were used to identify a T-DNA insertion at 1512 bp after the ATG of the cyp79B2 gene. The insertion is in the second exon of the cyp79B2 gene. Primer 79B3-5P (5′-TGTTCTATGCATGGACTGGT GGTCAACATG-3′; SEQ ID NO:3) and LB were used to identify a T-DNA insertion at 1425 bp after the ATG site of the cyp79B3 gene. The insertion is in the intron between the two exons of the cyp79B3 gene. The T-DNA insertion in the cyp79B3 gene is a tandem T-DNA insertion with LB flanking sequences at both ends of the T-DNA insertion. The insertions were confirmed by DNA sequencing of the PCR fragments generated with the LB primer and gene-specific primers.

Arabidopsis lines were grown in a greenhouse on the campus of the University of Illinois, Champaign, under 12 hours of light (˜100 uE/M²/sec) and dark. Plants were grown individually in 3.5-inch pots in L1 Sunshine mix. Seeds/seedlings were kept moist during germination, and plants were then watered daily to maintain soil moisture without saturating the soil. Plants were not fertilized. When inflorescences reached 6 cm, about 3.5 weeks, 50 plants of each ecotype were randomly clipped, leaving approximately 1 cm of inflorescence (comparable to natural mammalian herbivory (Siddappaji et al. (2013) Genetics 195:589-598).

At 6.5 weeks, 30 plants of Col-0 (15 clipped, 15 unclipped) were analyzed for indole glucosinolate concentration. Inflorescence material was taken from both clipped and unclipped plants. In addition, the level of indole glucosinolates were assessed from 30 plant (15 clipped and 15 unclipped) samples of the cyp79B2 cyp79B3 double mutant line to verify that there were in fact, undetectable levels of indole glucosinolates, consistent with the findings of Zhao et al. ((2002) Genes & Development 16:3100-3112). All samples were frozen in liquid nitrogen and stored at −80° C. prior to freeze-drying. Freeze-dried tissues were ground into a fine powder and stored at −20° C. prior to glucosinolate analysis. Glucosinolates were extracted from finely ground freeze-dried tissue, converted to desulphoglucosinolates with arylsulfatase and analyzed via high pressure liquid chromatography (HPLC). Freeze-dried powder (50 mg) and 0.5 mL of 70% methanol were added to 2.5 mL tubes and placed on a heating block at 95° C. for 10 minutes with frequent mixing. Samples were cooled on ice and 0.125 mL glucosinolabin was used as an internal standard and centrifuged at 3,000×g for 10 minutes. The supernatant was saved and the pellet was re-extracted with another 0.5 mL 70% methanol at 95° C. for 10 minutes and the two extracts were combined. Protein was subsequently precipitated with 0.15 mL of a 1:1 mixture of 1 M barium acetate and 1 M lead acetate and centrifuged at 12,000×g for 1 minute. Each sample was then loaded onto a column containing DEAE SEPHADEX® A-25 resin for desulfation via arylsulfatase for 18 hours and the remaining desulfo-GS eluted. Desulphoglucosinolates were separated on an HPLC system (Agilent 1100 HPLC system, with a G1311A bin pump, a G1322A vacuum degasser, a G1316A thermostatic column compartment, a G1315B diode array detector and an HP 1100 series G1313A autosampler) with a variable ultraviolet detector set at 229 nm wavelength. Elution of desulphoglucosinolates occurred over 45 minutes with a linear gradient of 0% to 20% acetonitrile in water with a flow rate of 1.0 mL/min. Glucosinolate concentration was established using glucosinalbin as an internal standard, a glucosinolate not found in A. thaliana. UV response factors for different glucosinolates were used as determined by Wathelet et al. ((2001) International Rapeseed Congress Technical Meeting, June 5-7, Poznan, Poland). Indole glucosinolates were estimated by adding the four indole glucosinolates observed in Col-0.

Upon plant senescence (8 weeks), remaining plants of both ecotypes were analyzed for fitness (70 plants per ecotype; 35 clipped, 35 unclipped). Fitness measures included the number of siliques, seeds per plant, and the average seed weight per plant. It has been shown that seeds are a good measure of plant fitness (Siddappaji et al. (2013) Genetics 195:589-598) as there are no significant differences in germination success between clipped and unclipped plants of A. thaliana. Total silique number for each plant was measured and total seed production was counted in 3 randomly selected siliques from each plant. Seeds per silique were averaged per ecotype and treatment, then multiplied by total silique number for each plant to obtain seed totals per plant. Average seed weights were measured by weighing 50 seeds per plant from 10 plants per ecotype x treatment group. Each weight measurement was then divided by the number of seeds to yield average seed weight for each ecotype x treatment group. Additionally, rosette diameter was measured at the time of senescence for Col-0 and cyp79B2 cyp79B3.

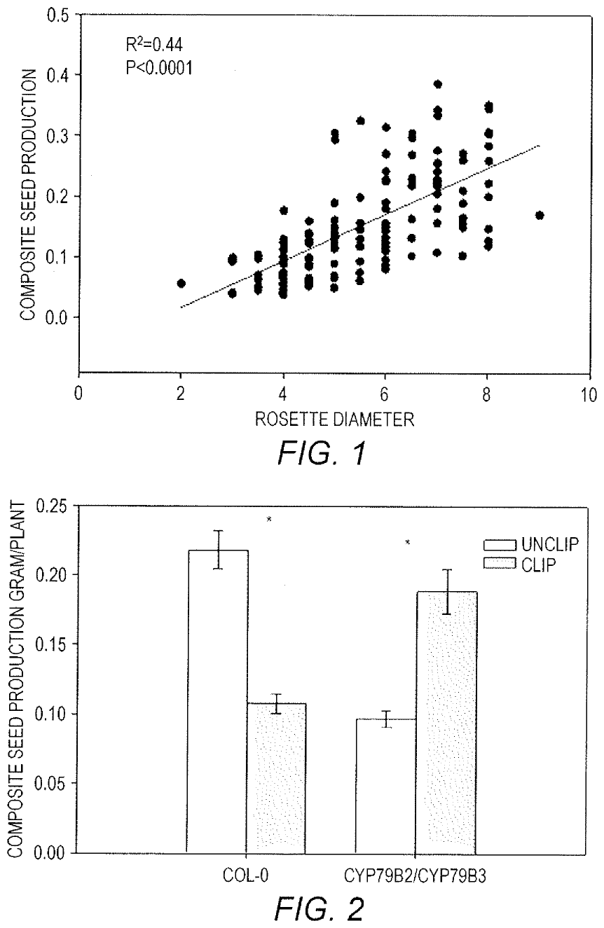

Potential differences in composite seed production were assessed using an analysis of variance and Type III sums of squares with two treatment factors (genotype and clipping). Rosette diameter was used as a covariate to adjust for differences in plant size. In addition, total seed production on rosette diameter of A. thaliana was regressed for both Col-O and cyp79B2 cyp79B3 double mutant lines to justify the use of rosette diameter as an appropriate covariate in the model (see FIG. 1). Differences among treatments within genotypes were determined using Tukey pairwise comparisons. Glucosinolate production, between clipped and unclipped Col-0 plants, was assessed using a two-sample t-test. Statistical analyses were conducted in Systat (version 13; Systat, Chicago, Illinois, USA).

### Example 2: Results of Clipping of Arabidopsis thaliana Col-0 and Col-0 Double Mutant cyp79B2 cyp79B3

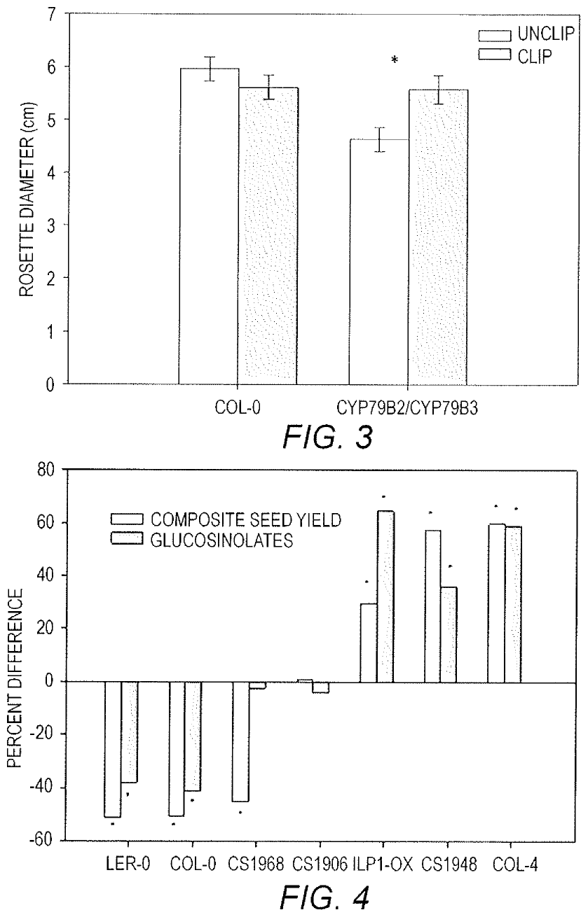

As shown in FIG. 2, genotypes differed in composite seed production in their response to removal of the apical meristem, i.e., clipping (p<0.001; Table 1). Specifically, a post hoc Tukey test revealed Col-0 significantly undercompensated with a 50.5% decrease in composite seed yield (grams/plant) (when comparing damaged plants to undamaged controls, p<0.001). In contrast, the cyp79B2 cyp79B3 double mutant with reduced glucosinolate content significantly overcompensated (p<0.001) following removal of the apical meristem with a 94% increase in composite seed yield (FIG. 2). Results remained the same following adjustment with rosette size run as a covariate to adjust composite seed yield means (FIG. 3). Results show that there is a linear relationship between composite seed yield and rosette size justifying rosette size as an appropriate covariate in this analysis (FIG. 1).

TABLE 1

| | Type III | | Mean | | |
|---|---|---|---|---|---|
| Source | SS | Df | Square | F-ratio | p-Value |
| Rosette | 0.306 | 1 | 0.306 | 137.793 | 0.000 |
| Genotype | 0.000 | 1 | 0.000 | 0.116 | 0.734 |
| Clipping | 0.012 | 1 | 0.012 | 5.343 | 0.022 |
| Genotype × | 0.191 | 1 | 0.191 | 85.977 | 0.000 |
| Clipping | | | | | |
| Error | 0.296 | 133 | 0.002 | | |

Genotypes also differed in indole glucosinolate concentrations in their response to removal of the apical meristem (p<0.001). Unclipped Col-0 plants produced on average 1.35 μmol g⁻¹ of total indole glucosinolates, whereas clipped Col-0 displayed a 41.48% decrease (producing 0.79 μmol g⁻¹; t=2.88, df=2.28 p<0.008) in indole glucosinolates following removal of the apical meristem. It was also confirmed that the knockout mutants did not produce any indole glucosinolates.

It has been proposed that there should be a trade-off between plant resistance and tolerance given that resources are limited. However, it has been shown that both resistance and tolerance are positively and causally entwined within the same molecular pathway (the oxidative pentose-phosphate pathway leading directly into the shikimate pathway) (Siddappaji et al. (2013) Genetics 195:589-598). By measuring glucosinolate levels and seed production (the measure of tolerance herein) following the removal of apical dominance in Arabidopsis thaliana (ranging the entire spectrum of seed production from undercompensation to equal compensation to overcompensation), it has now been shown that there is a positive association between tolerance and induced resistance. For example, plants that undercompensated for seed production following the removal of apical dominance also showed a decrease in glucosinolate production, while plants that equally compensated for seed production equally compensated for glucosinolate production, and plants that overcompensated for seed production overcompensated for glucosinolate production (FIG. 4; from Mesa et al. (2017) Ecology 98:2528-2537). Thus, these results indicate that selection favors intermediate levels of both resistance and tolerance, questioning the generality of the assumptions behind the trade-off hypothesis, i.e., when plants experience herbivory from both generalist herbivores that are susceptible to resistance traits and specialist herbivores that can circumvent resistance traits, selection should favor intermediate levels of tolerance and resistance.

Despite a positive relationship between tolerance and resistance, the results herein show that there is still a predictable cost/trade-off in maintaining them. The results show that knocking out the indole glucosinolate biosynthetic pathway via a cyp79B2 cyp79B3 double mutant in a Col-0 background results in plants overcompensating after tissue damage, with clipped plants having significantly higher total seed production compared to the undercompensating wild-type Col-0. As both resistance and tolerance are controlled via the oxidative pentose-phosphate pathway (Scholes & Paige (2014) Molecular Ecology 23:4862-4870; Mesa et al. (2017) Ecology 98:2528-2537) knocking out production of indole glucosinolates should allow more resources to be shunted towards tolerance.

However, knockout mutants for indole glucosinolate biosynthesis also have reduced levels of the growth regulator indole-3-acetic acid (IAA), as indole-3-acetaldoxime (IAOx) is used as an intermediate of both indole glucosinolates and IAA biosynthesis. IAOx is reduced by knocking out the Cytochrome P450 enzymes cyp79B2 and cyp79B3, hence reducing IAA (Zhao et al. (2002) Genes & Development 16:3100-3112). IAA is the main auxin in plants, regulating growth and developmental processes such as cell division and elongation, tissue differentiation, and apical dominance. Effects of the downregulation of auxin can be seen in this experiment as rosette diameters of unclipped plants of cyp79B2 cyp79B3 were significantly smaller than wild-type plants.

In contrast, clipped cyp79B2 cyp79B3 double knockout rosettes were as large as the wild-type treatments and these plants overcompensated compared to unclipped cyp79B2 cyp79B3 double knockout plants (note, the same statistical outcome was observed in terms of seed production whether adjusted for unclipped cyp knockout plant sizes or not in this experiment). It was also clear that the clipped cyp79B2 cyp79B3 double knockout plants overcompensated when comparing them to the clipped wild-type plants, consistent with an increase in resources toward compensation given that one of two major chemical pathways was knocked out.

The combination of clipping the cyp79B2 cyp79B3 knockout, along with the reduction in resistance, likely lead to an increase in IAA production through the process of endoreduplication (via an increase in gene expression). Zhao et al. ((2002) Genes & Development 16:3100-3112) have shown that alternative pathways of IAA production remain active and these alternative pathways are likely increased through the process of endoreduplication triggered by the removal of apical dominance. Notably, cell cycle values for clipped cyp knockout plants were significantly higher than for clipped wild-type plants (0.500±0.47 vs. 0.376±0.057, t=4.29, df=13, p=0.001). Thus, the increase in fitness not only involves a reduction in resistance but in this case an increase in the level of IAA through endoreduplication following the removal of apical dominance. It is important to note that the clipped wild-type plants undercompensated and were not affected by a reduction in IAA, suggesting, at least in part, that it is the reduction in resistance that contributes to the increase in reproductive success.

Mechanistically, bypassing the shikimate pathway, may lead to an increase in the production of glucose-6-phosphate-dehydrogenase-1 (G6PD1), that plays a key role in compensation and primary metabolism (Siddappaji et al. (2013) Genetics 195:589-598), ultimately leading to an increase in plant growth and reproduction. There is also the possibility that blockage of the shikimate pathway increases the availability of translational machinery (e.g., ribosomes) leading to an increase in the compensatory response through endoreduplication.

Despite a positive relationship between tolerance and resistance in Arabidopsis thaliana, the results herein indicate that plant resistance carries a significant cost in tolerance, suppressing the degree of compensation.

1-6. (canceled)

7: A method of increasing one or more agronomic traits of a plant comprising inhibiting or reducing the amount of one or more chemical defense compounds produced by the plant to produce a modified plant and removing the shoot apical meristem of the modified plant, thereby increasing one or more agronomic traits of the plant as compared to an unmodified plant,
wherein the one or more chemical defense compounds are glucosinolates and the modified plant carries one or more mutations in a cytochrome P450 gene encoding a cytochrome P450 that catalyzes the synthesis of one or more glucosinolates such that the modified plant produces less of the glucosinolates compared a corresponding unmodified plant,
wherein the one or more agronomic traits is selected from the group consisting of seed number, pod number, seed biomass, pod biomass, seed oil content, seed protein content, plant biomass, root biomass, leaf biomass, tuber biomass, fruit biomass, stem biomass, and wood biomass, and
wherein the plant is selected from the group consisting of soybean, canola, rape, potato, sunflower, flax, safflower, and camelina.

8-10. (canceled)

11: The method of claim 7, wherein the step of removing the shoot apical meristem of plants is selected from the group consisting of physical removal by cutting and chemical removal.

12-18. (canceled)


SEQUENCE LISTING (3 sequences)

SEQ ID NO: 1 - DNA, 30 bp, synthetic construct
  source 1..30: mol_type = other DNA
tggacaagtatcatgacccaatcatccacg

SEQ ID NO: 2 - DNA, 30 bp, synthetic construct
  source 1..30: mol_type = other DNA
ggcaatcagctgttgcccgtctcactggtg

SEQ ID NO: 3 - DNA, 30 bp, synthetic construct
  source 1..30: mol_type = other DNA
tgttctatgcatggactggtggtcaacatg